(19) United States
(12) Patent Application Publication
(10) Pub. No.: US 2026/0130837 A1
(43) Pub. Date: May 14, 2026

(54) SPF BOOSTER AND SUNCARE FORMULATIONS INCLUDING SAME

(71) Applicants: Dow Silicones Corporation, Midland, MI (US); Rohm and Haas Company, Collegeville, PA (US)

(72) Inventors: Margery Cortes-Clerget, Midland, MI (US); Marc-Andre Courtemanche, Midland, MI (US); Wenjun Xu, Eagleville, PA (US); Helene Dihang, Taisnières-sur-Hon (FR); Jason Vogel, Midland, MI (US)

(21) Appl. No.: 19/121,148

(22) PCT Filed: Oct. 12, 2023
(86) PCT No.: PCT/US2023/076637

§ 371 Date: Apr. 15, 2025

Related U.S. Application Data
(60) Provisional application No. 63/416,950, filed on Oct. 18, 2022.

(51) Int. Cl. A61K 8/73; A61K 8/35; A61K 8/37; A61K 8/40; A61Q 17/04

(52) U.S. Cl. CPC A61K 8/73; A61K 8/35; A61K 8/37; A61K 8/40; A61Q 17/04

(57) ABSTRACT

An SPF booster is provided comprising a functionalized maltodextrin comprising a maltodextrin base polymer functionalized with —Si(R¹)₃ groups; wherein each R¹ is independently a C₁₋₁₀ linear or branched, saturated alkyl group; wherein the maltodextrin base polymer has a dextrose equivalent, DE, of 1 to 15; wherein the functionalized maltodextrin has a degree of substitution of —Si(R¹)₃ groups, DS, of 1.7 to 3; and wherein the functionalized maltodextrin is free of vinylic carbon. Also provided are suncare formulations containing same and methods of using same.

The present invention relates to an SPF booster and suncare formulations comprising the same. In particular, the present invention relates to an suncare booster, comprising: a functionalized maltodextrin comprising a maltodextrin base polymer functionalized with —Si(R¹)₃ groups; wherein each R¹ is independently a C₁₋₁₀ linear or branched, saturated alkyl group; wherein the maltodextrin base polymer has a dextrose equivalent, DE, of 1 to 15; wherein the functionalized maltodextrin has a degree of substitution of —Si(R¹)₃ groups, DS, of 1.7 to 3; and wherein the functionalized maltodextrin is free of vinylic carbon; and to suncare formulations including same.

The damaging effects of sunlight on human skin are well documented. Six percent of the solar energy reaching the Earth's surface is ultraviolet (UV) radiation having a wavelength of 290 to 400 nm. This radiation is divided into two components: (i) low energy UVA radiation having a wavelength of 320 to 400 nm and (ii) high energy UVB radiation having a wavelength of 290 to 320 nm. While the UV portion of solar energy is relatively small, it induces nearly 99% of all the side effects from sunlight exposure. High energy UVB radiation, for example, is responsible for producing sunburn, appearance of skin aging and skin cancer. Low energy UVA radiation, for example, is responsible for inducing direct tanning and erythema (abnormal redness) of the skin and contributes to the appearance of skin aging.

By avoiding direct exposure to sunlight, individuals can avoid the serious effects caused by exposure to UV radiation. However, because of the nature of their work, it is challenging for some people to avoid such exposure. In addition, some people voluntarily expose their skin to the sun, e.g., to tan. Therefore, protection against the harmful effects of the sun is important.

Protection from the harmful effects of UV radiation exposure is available in the form of both topically applied formulations containing at least one physical UV blocker, or at least one chemical UV absorber, or combinations thereof. Physical blockers include active ingredients such as, titanium dioxide, zinc oxide and red petrolatum. Chemical absorbers include active ingredients, such as, paraaminobenzoic acid (more commonly known as PABA), which are generally transparent when applied and act by absorbing UV radiation, offering selective protection against certain UV wave bands, depending on the absorption spectrum of the active ingredient in the formulation.

The effectiveness of a given sunscreen formulation is assessed by how well it protects the skin in terms of a Sun Protection Factor (SPF) which is defined as the ratio of the amount of energy required to produce a minimal erythema on sunscreen protected skin to the amount of energy required to produce the same level of erythema on unprotected skin.

A number of the chemical absorbers and physical blockers, herein after referred to as “UV radiation absorbing agents,” typically used in sunscreen formulations reportedly have perceived adverse toxicological effects, perceived negative sensory effects and perceived negative environmental impacts, which discourage some people from using sunscreens. Therefore, it is desirable to reduce the level of UV radiation absorbing agents present in sunscreen formulations without reducing the SPF protection. Accordingly, a variety of SPF boosters have been developed for use in water based sunscreen formulations to reduce the level of UV radiation absorbing agents without a reduction in the SPF protection provided.

To that end, an approach to improving UV radiation absorption of a composition containing at least one UV radiation absorbing agent through the incorporation of a voided latex particle is disclosed in U.S. Pat. No. 5,663,213 to Jones et al. Jones et al. disclose a method for improving UV radiation absorption of a composition, comprising: adding to said composition from about 0.1 weight percent to about 50 weight percent of latex particles, based on total weight non-volatiles, wherein the composition comprises at least one UV radiation absorbing agent, wherein the latex particles contain a void and have a particle size of from about 100 nm to about 380 nm, and wherein the latex particles are added to increase the UV radiation absorption of the composition.

Notwithstanding, alcohol based sunscreen products (e.g., sprays) account for up to 50% of the market. High alcohol content (e.g., >60 wt % ethanol) sunscreens are a popular format for which many conventional SPF booster offerings are unsuited.

To achieve desired SPF ratings in organic carrier based (sprayable) suncare formulations, conventional formulations incorporate high levels of expensive UV absorbing agents. While increasing the cost of the formulations, the incorporation of high levels of UV absorbing agents also negatively impair the aesthetics of these conventional formulations often imparting a tacky or gritting sensation on the skin.

Accordingly, there remains a need for new alcohol based suncare formulations that provide an effective SPF rating while reducing the necessary incorporation level of UV absorbing agents and enhancing the aesthetics of the formulation during use.

The present invention provides an SPF booster, comprising: a functionalized maltodextrin comprising a maltodextrin base polymer functionalized with —Si(R¹)₃ groups; wherein each R¹ is independently a C₁₋₁₀ linear or branched, saturated alkyl group; wherein the maltodextrin base polymer has a dextrose equivalent, DE, of 1 to 15; wherein the functionalized maltodextrin has a degree of substitution of —Si(R¹)₃ groups, DS, of 1.7 to 3; and wherein the functionalized maltodextrin is free of vinylic carbon.

The present invention provides a suncare formulation, comprising: a dermatologically acceptable organic carrier; a UV radiation absorbing agent; and an SPF booster, comprising a functionalized maltodextrin comprising a maltodextrin base polymer functionalized with —Si(R¹)₃ groups; wherein each R¹ is independently a C₁₋₁₀ linear or branched, saturated alkyl group; wherein the maltodextrin base polymer has a dextrose equivalent, DE, of 1 to 15; wherein the functionalized maltodextrin has a degree of substitution of —Si(R¹)₃ groups, DS, of 1.7 to 3; and wherein the functionalized maltodextrin is free of vinylic carbon.

The present invention provides a method of protecting skin from exposure to the sun, comprising: providing a suncare formulation of the present invention, and applying the suncare formulation to skin.

### DETAILED DESCRIPTION

We have surprisingly found an SPF booster comprising a functionalized maltodextrin comprising a maltodextrin base polymer functionalized with —Si(R¹)₃ groups; wherein each R¹ is independently a C₁₋₁₀ linear or branched, saturated alkyl group; wherein the maltodextrin base polymer has a dextrose equivalent, DE, of 1 to 15; wherein the functionalized maltodextrin has a degree of substitution of —Si(R¹)₃ groups, DS, of 1.7 to 3; and wherein the functionalized maltodextrin is free of vinylic carbon. The SPF booster of the present invention is a biobased and biodegradable material. Moreover, we have surprisingly found that the SPF booster of the present invention is provides SPF boosting when incorporated into an alcohol based sunscreen formulation with desirable aesthetic characteristics.

Unless otherwise indicated, ratios, percentages, parts, and the like are by weight.

The term “dextrose equivalent, DE” as used herein and in the appended claims refers to the degree of starch hydrolysis, specifically, the reducing value of a starch hydrolysate material compared to the reducing value of an equal weight of dextrose, expressed as percent, dry basis, as measured by the Lane and Eynon method described in Standard Analytical Method E-26, Corn Refiners Association, 6^{th} edition, 1977, E-26, pp. 1-3. For example, a maltodextrin with a DE of 10 would have 10% of the reducing power of dextrose which has a DE of 100.

The term “vinylic carbon” as used herein and in the appended claims refers to a carbon that is involved in a double bond with another carbon.

The term “free of vinylic carbon” as used herein and in the appended claims in reference to the functionalized maltodextrin means that the functionalized maltodextrin contains less than the detectable limit of vinylic carbon.

The term “dermatologically acceptable” as used herein and in the appended refers to ingredients typically used in personal care compositions, and is intended to underscore that materials that are toxic when present in the amounts typically found in personal care compositions are not contemplated as part of the present invention.

The term “aesthetic characteristics” as used herein and in the appended claims in reference to visual and tactile sensory properties (e.g., smoothness, tack, lubricity, texture, color, clarity, turbidity, uniformity).

Preferably, the suncare formulation of the present invention is provided in a product form selected from the group consisting of a cream, a non-aqueous solution, an oil, an ointment, a paste, a gel, a lotion, a milk, a foam, a stick and a suspension. More preferably, the suncare formulation of the present invention is provided as a non-aqueous solution. Most preferably, the suncare formulation of the present invention is formulated for application to skin using a mechanical device (e.g., manual pump spray containers, squeeze bottles) or a pressurized aerosol container (e.g., bag-on-nozzle container, pressurized can) to generate a spray.

Preferably, the SPF booster of the present invention, comprises a functionalized maltodextrin comprising a maltodextrin base polymer functionalized with —Si(R¹)₃ groups; wherein each R¹ is independently a C₁₋₁₀ linear or branched, saturated alkyl group (preferably, a methyl group, an ethyl group, a propyl group, a butyl group and a pentyl group; more preferably, a methyl group, an ethyl group, a propyl group and a butyl group; still more preferably, a methyl group, an ethyl group and a propyl group; yet more preferably, a methyl group and an ethyl group; most preferably, a methyl group); wherein the maltodextrin base polymer has a dextrose equivalent, DE, of 1 to 15 (preferably, 1 to 12; more preferably, 1 to 10; most preferably, 1 to 7); wherein the functionalized maltodextrin has a degree of substitution of —Si(R¹)₃ groups, DS, of 1.7 to 3 (preferably, 1.8 to 3.0; more preferably, 2 to 2.8; most preferably, 2.1 to 2.55); and wherein the functionalized maltodextrin is free of vinylic carbon. More preferably, the SPF booster of the present invention, comprises a functionalized maltodextrin comprising a maltodextrin base polymer functionalized with —Si(R¹)₃ groups; wherein the —Si(R¹)₃ groups are linked to the maltodextrin base polymer through a C—O—Si bond; wherein each R¹ is independently a C₁₋₁₀ linear or branched, saturated alkyl group (preferably, a methyl group, an ethyl group, a propyl group, a butyl group and a pentyl group; more preferably, a methyl group, an ethyl group, a propyl group and a butyl group; still more preferably, a methyl group, an ethyl group and a propyl group; yet more preferably, a methyl group and an ethyl group; most preferably, a methyl group); wherein the maltodextrin base polymer has a dextrose equivalent, DE, of 1 to 15 (preferably, 1 to 12; more preferably, 1 to 10; most preferably, 1 to 7); wherein the functionalized maltodextrin has a degree of substitution of —Si(R¹)₃ groups, DS, of 1.7 to 3 (preferably, 1.8 to 3.0; more preferably, 2 to 2.8; most preferably, 2.1 to 2.55); and wherein the functionalized maltodextrin is free of vinylic carbon.

Preferably, the suncare formulation of the present invention comprises: a dermatologically acceptable organic carrier (preferably, 10 to 98 wt % (more preferably, 25 to 92 wt %; still more preferably, 35 to 85 wt %; most preferably, 40 to 80), based on weight of the suncare formulation, of the dermatologically acceptable organic carrier); a UV radiation absorbing agent (preferably, 0.1 to 70 wt % (more preferably, 5 to 65 wt %; still more preferably, 7.5 to 60 wt %; most preferably, 10 to 55 wt %), based on weight of the suncare formulation, of the UV radiation absorbing agent); and an SPF booster (preferably, 0.1 to 70 wt % (more preferably, 1 to 15 wt %; still more preferably, 1.5 to 10 wt %; most preferably, 2 to 6 wt %), based on weight of the suncare formulation, of the SPF booster); wherein the SPF booster, comprises a functionalized maltodextrin comprising a maltodextrin base polymer functionalized with —Si(R¹)₃ groups; wherein each R¹ is independently a C₁₋₁₀ linear or branched, saturated alkyl group (preferably, a methyl group, an ethyl group, a propyl group, a butyl group and a pentyl group; more preferably, a methyl group, an ethyl group, a propyl group and a butyl group; still more preferably, a methyl group, an ethyl group and a propyl group; yet more preferably, a methyl group and an ethyl group; most preferably, a methyl group); wherein the maltodextrin base polymer has a dextrose equivalent, DE, of 1 to 15 (preferably, 1 to 12; more preferably, 1 to 10; most preferably, 1 to 7); wherein the functionalized maltodextrin has a degree of substitution of —Si(R¹)₃ groups, DS, of 1.7 to 3 (preferably, 1.8 to 3.0; more preferably, 2 to 2.8; most preferably, 2.1 to 2.55); and wherein the functionalized maltodextrin is free of vinylic carbon. More preferably, the suncare formulation of the present invention comprises: a dermatologically acceptable organic carrier (preferably, 10 to 98 wt % (more preferably, 25 to 92 wt %; still more preferably, 35 to 85 wt %; most preferably, 40 to 80), based on weight of the suncare formulation, of the dermatologically acceptable organic carrier); a UV radiation absorbing agent (preferably, 0.1 to 70 wt % (more preferably, 5 to 65 wt %; still more preferably, 7.5 to 60 wt %; most preferably, 10 to 55 wt %), based on weight of the suncare formulation, of the UV radiation absorbing agent); and an SPF booster (preferably, 0.1 to 70 wt % (more preferably, 1 to 15 wt %; still more preferably, 1.5 to 10 wt %; most preferably, 2 to 6 wt %), based on weight of the suncare formulation, of the SPF booster); wherein the SPF booster, comprises a functionalized maltodextrin comprising a maltodextrin base polymer functionalized with —Si(R¹)₃ groups; wherein the —Si(R¹)₃ groups are linked to the maltodextrin base polymer through a C—O—Si bond; wherein each R¹ is independently a C₁₋₁₀ linear or branched, saturated alkyl group (preferably, a methyl group, an ethyl group, a propyl group, a butyl group and a pentyl group; more preferably, a methyl group, an ethyl group, a propyl group and a butyl group; still more preferably, a methyl group, an ethyl group and a propyl group; yet more preferably, a methyl group and an ethyl group; most preferably, a methyl group); wherein the maltodextrin base polymer has a dextrose equivalent, DE, of 1 to 15 (preferably, 1 to 12; more preferably, 1 to 10; most preferably, 1 to 7); wherein the functionalized maltodextrin has a degree of substitution of —Si(R¹)₃ groups, DS, of 1.7 to 3 (preferably, 1.8 to 3.0; more preferably, 2 to 2.8; most preferably, 2.1 to 2.55); and wherein the functionalized maltodextrin is free of vinylic carbon.

Preferably, the maltodextrin base polymer has a dextrose equivalent, DE, of 1 to 15 (preferably, 1 to 12; more preferably, 1 to 10; most preferably, 1 to 7). More preferably, the maltodextrin base polymer has a dextrose equivalent, DE, of 1 to 15 (preferably, 1 to 12; more preferably, 1 to 10; most preferably, 1 to 7); wherein the maltodextrin base polymer is a straight or branched chain maltodextrin polymer comprising a plurality of glucose structural units. Most preferably, the maltodextrin base polymer has a dextrose equivalent, DE, of 1 to 15 (preferably, 1 to 12; more preferably, 1 to 10; most preferably, 1 to 7); wherein the maltodextrin base polymer is a straight or branched chain maltodextrin polymer comprising a plurality of glucose structural units; wherein 90 to 100 mol % (preferably, 92 to 100 mol %; more preferably, 93 to 100 mol %; most preferably, 94.5 to 100 mol %) of the glucose structural units are connected by α-1,4 linkages and 0 to 10 mol % (preferably, 0 to 8 mol %; more preferably, 0 to 7 mol %; most preferably, 0 to 5.5 mol %) of the glucose structural units are connected by α-1,6 linkages.

Preferably, the maltodextrin base polymer contains less than 0.01 wt %, based on weight of the maltodextrin base polymer, of alternan. More preferably, the maltodextrin base polymer contains less than 0.001 wt %, based on weight of the maltodextrin base polymer, of alternan. Most preferably, the maltodextrin base polymer contains less than the detectable limit of alternan.

Preferably, <0.1 mol % (preferably, <0.01 mol %; more preferably, <0.001 mol %; most preferably, <detectable limit) of the glucose structural units in the maltodextrin base polymer are connected by β-1,4 linkages.

Preferably, <0.1 mol % (preferably, <0.01 mol %; more preferably, <0.001 mol %; most preferably, <detectable limit) of the glucose structural units in the maltodextrin base polymer are connected by β-1,3 linkages.

Preferably, the suncare formulation of the present invention, comprises 10 to 98 wt % (preferably, 25 to 92 wt %; more preferably, 35 to 85 wt %; most preferably, 40 to 80), based on weight of the suncare formulation, of a dermatologically acceptable organic carrier. More preferably, the suncare formulation of the present invention, comprises 10 to 98 wt % (preferably, 25 to 92 wt %; more preferably, 35 to 85 wt %; most preferably, 40 to 80), of a dermatologically acceptable organic carrier; wherein the dermatologically acceptable organic carrier is selected from the group consisting of glycols (e.g., ethylene glycol, propylene glycol, butylene glycol, pentylene glycol, hexylene glycol, dipropylene glycol, ethoxydiglycol); C₁₋₁₀ straight or branched chain alcohols (e.g., methyl alcohol, ethyl alcohol, propyl alcohol, isopropyl alcohol, butyl alcohol, 2-butoxyethanol); ketones (e.g., acetone); acetates (e.g., methyl acetate); butyl cellusolve; dimethicones; polydimethylsiloxanes; alkanes (e.g., isododecane, isohexane); alkanoates (e.g., methyl undecanoate), dermatologically acceptable hydrophobic ester oils (e.g., caprylic capric triglycerides); dicaprylyl carbonate; alkyl benzoates (e.g., C₁₂₋₁₅ alkyl benzoate); hemisqualane; dioctylether; keto acids (e.g., levulinic acid) and mixtures thereof. Still more preferably, the suncare formulation of the present invention, comprises 10 to 98 wt % (preferably, 25 to 92 wt %; more preferably, 35 to 85 wt %; most preferably, 40 to 80), of a dermatologically acceptable organic carrier; wherein the dermatologically acceptable organic carrier is selected to be capable of evaporating upon application of the suncare formulation to the skin. Yet more preferably, the suncare formulation of the present invention, comprises 10 to 98 wt % (preferably, 25 to 92 wt %; more preferably, 35 to 85 wt %; most preferably, 40 to 80), of a dermatologically acceptable organic carrier; wherein the dermatologically acceptable organic carrier includes a C₁₋₄ straight or branched chain alcohol (e.g., methyl alcohol, ethyl alcohol, propyl alcohol, isopropyl alcohol, butyl alcohol)(preferably, wherein the alcohol is a denatured alcohol). Most preferably, the suncare formulation of the present invention, comprises 10 to 98 wt % (preferably, 25 to 92 wt %; more preferably, 35 to 85 wt %; most preferably, 40 to 80), of a dermatologically acceptable organic carrier; wherein the dermatologically acceptable organic carrier includes a specifically denatured ethyl alcohol (e.g., INCI: SD alcohol 40-B; INCI: Alcohol Denat.).

Preferably, the suncare formulation of the present invention, comprises 0.1 to 70 wt % (preferably, 5 to 65 wt %; more preferably, 7.5 to 60 wt %; most preferably, 10 to 55 wt %), based on weight of the suncare formulation, of a UV radiation absorbing agent. More preferably, the suncare formulation of the present invention, comprises 0.1 to 70 wt % (preferably, 5 to 65 wt %; more preferably, 7.5 to 60 wt %; most preferably, 10 to 55 wt %), of a UV radiation absorbing agent; wherein the UV radiation absorbing agent is selected from the group including physical blockers (e.g., red petrolatum, titanium dioxide, zinc oxide); chemical absorbers (e.g., 1-(4-methoxyphenol)-3-(4-tert-butylphenyl)propane-1,3-dione (INCI: avobenzone), 2-hydroxy-4-methoxybenzophenone (INCI: oxybenzone); dioxybenzone; sulisobenzone; menthyl anthranilate; para-aminobenzoic acid; amyl paradimethylaminobenzoic acid; octyl para-dimethylaminobenzoate; ethyl 4-bis (hydroxypropyl) para-aminobenzoate; polyethylene glycol (PEG-25) para-aminobenzoate; ethyl 4-bis(hydroxypropyl) aminobenzoate; diethanolamine para-methyoxycinnamate; 2-ethoxyethyl para-methoxycinnamate; ethylhexyl para-methoxycinnamate; octyl para-methoxycinnamate; isoamyl para-methoxycinnamate; 2-ethylhexyl-2-cyano-3,3-diphenyl-acrylate; 2-ethylhexyl-2-cyano-3,3-diphenyl-2-propenoate (INCI: octocrylene); 2-ethylhexyl 2-hydroxybenzoate (INCI: octisalate); homomenthyl salicylate; glyceryl aminobenzoate; triethanolamine salicylate; digalloyl trioleate; lawsone with dihydroxyacetone; 2-phenylbenzimidazole-5-sulfonic acid; 4-methylbenzylidine camphor; avobenzone; triazines; benzotriazoles; vinyl group-containing amides; cinnamic acid amides; sulfonated benzimidazoles); 3,3,5-trimethylcyclohexyl 2-hydroxybenzoate (INCI: Homosalate)); and mixtures thereof. Still more preferably, the suncare formulation of the present invention, comprises 0.1 to 70 wt % (preferably, 5 to 65 wt %; more preferably, 7.5 to 60 wt %; most preferably, 10 to 55 wt %), of a UV radiation absorbing agent, wherein the UV radiation absorbing agent comprises a mixture of UV radiation absorbing agents. Yet more preferably, the suncare formulation of the present invention, comprises 0.1 to 70 wt % (preferably, 5 to 65 wt %; more preferably, 7.5 to 60 wt %; most preferably, 10 to 55 wt %), of a UV radiation absorbing agent, wherein the UV absorbing agent is a mixture of UV absorbing agents including at least one of 1-(4-methoxyphenol)-3-(4-tert-butylphenyl)propane-1,3-dione (INCI: avobenzone); 2-ethylhexyl 2-hydroxybenzoate (INCI: octisalate); 2-ethyhexyl-2-cyano-3,3-diphenyl-2-propenoate (INCI: octocrylene); 2-hydroxy-4-methoxybenzophenone (INCI: oxybenzone) and 3,3,5-trimethylcyclohexyl 2-hydroxybenzoate (INCI: Homosalate). Most preferably, the suncare formulation of the present invention, comprises 0.1 to 70 wt % (preferably, 5 to 65 wt %; more preferably, 7.5 to 60 wt %; most preferably, 10 to 55 wt %), of a UV radiation absorbing agent, wherein the UV absorbing agent is a mixture of UV absorbing agents including 1-(4-methoxyphenol)-3-(4-tert-butylphenyl)propane-1,3-dione (INCI: avobenzone); 2-ethylhexyl 2-hydroxybenzoate (INCI: octisalate); 2-ethyhexyl-2-cyano-3,3-diphenyl-2-propenoate (INCI: octocrylene) and 3,3,5-trimethylcyclohexyl 2-hydroxybenzoate (INCI: Homosalate).

Preferably, the suncare formulation of the present invention has an SPF of ≥10 (more preferably, ≥20; still more preferably, ≥25; yet more preferably, ≥30; most preferably, ≥35) (preferably, wherein the SPF of the formulation is measured as described in the Examples).

Preferably, the suncare formulation of the present invention contains less than 5 wt % (more preferably, <4 wt %; still more preferably, <3 wt %; yet more preferably, <2 wt %; most preferably, <1 wt %) water.

Preferably, the suncare formulation of the present invention, further comprises an optional additive. More preferably, the suncare formulation of the present invention, further comprises an optional additive, wherein the optional additive is selected from the group consisting of film forming agent, water proofing agents, emollients, preservatives, antioxidants, fragrances, humectants, rheology modifiers, aesthetic modifiers, propellants, Vitamins, skin protectants, oils, emulsifiers, surfactants, pearlizers, consistency factors, thickeners, super fatting agents, stabilizers, polymers, silicone compounds, fats, waxes, lectins, phospholipids and mixtures thereof.

Preferably, the suncare formulation of the present invention, further comprises a film forming agent. More preferably, the suncare formulation of the present invention, further comprises 0.1 to 10 wt % (preferably, 0.5 to 9 wt %; more preferably, 0.7 to 5 wt %) of a film forming agent. Most preferably, the suncare formulation of the present invention, further comprises 0.1 to 10 wt % (preferably, 0.5 to 9 wt %; more preferably, 0.7 to 5 wt %) of a film forming agent; wherein the film forming agent is selected to provide a film barrier upon application of the suncare formulation of the present invention to skin. The purpose of the film barrier is to help maintain the UV radiation absorbing agents on the skin following immersion in water.

Preferred film forming agents include petrolatum; emollient esters (e.g., C₈₋₂₄ alkyl triglyceride (preferably, an aliphatic C₁₂₋₂₄ alkyl triglyceride; more preferably, an aliphatic C_{>12-24} alkyl triglyderide; most preferably, caprylic/capric triglycerides); lanolin derivatives (e.g., acetylated lanolins); superfatted oils; silicone gum; silicone elastomer; silicone resin; phenyl functionalized silicones; silicone acrylates; dimethicone derivatives; natural and synthetic oils; fatty acids; fatty alcohols; waxes; acrylic copolymers; polyamides; polyesters; polysaccharides; acrylate polymers and mixtures thereof.

Acrylic copolymers include acrylamide/acrylic copolymers (e.g., Dermacryl® 79 (INCI: Acrylates/Octyacrylamide copolymer) available from National Starch and Chemical); acrylates copolymers (e.g., EPITAX™ 66 powder water resistant polymer (INCI: acrylates copolymer) available from The Dow Chemical Company).

Certain emollients also exhibit film forming functionality by providing a water-resistant barrier on skin. Emollients with film forming behavior include butyloctyl salicylate (e.g., HallBrite® BHB available form HallStar); fatty acids (e.g., oleic, stearic); fatty alcohols (e.g., cetyl, hexadecyl); esters (e.g., 2,2-dimethyl-1,3-propanediyl diheptanoate (INCI: neopentyl glycol diheptanoate)); alkanes (e.g., mineral oil); ethers (e.g., polyoxypropylene butyl ethers, polyoxypropylene cetyl ethers); natural oils and synthetic oils (including silicone oils).

Preferred propellants for use in the suncare formulation of the present invention, include methane, ethane, propane, isobutane, n-butane, hexane, heptane, dimethyl ether, diethyl ether, fluoro containing materials (e.g., 1,1-difluoroethane, ethyl perfluoroisobutyl ether, ethyl perfluorobutyl ether, methyl perfluoroisobutyl ether, methyl perfluorobutyl ether) and mixtures thereof. Preferred fluoro containing propellants include Cosmetic Fluid CF-76 (INCI designation: ethyl perfluorobutyl ether/ethyl perfluoroisobutyl ether) and Cosmetic Fluid CF-61 (INCI designation: methyl perfluorobutyl ether/methyl perfluoroisobutyl ether).

The suncare formulation of the present invention are useful for the protection of skin. Preferably, the suncare formulations of the present invention are useful for the protecting skin from UV damage from exposure to the sun. The suncare formulations of the present invention also preferably provide moisturization to the skin, prevention and treatment of dry skin, protection of sensitive skin, improvement of skin tone and texture, masking imperfections, and inhibition of trans-epidermal water loss. Thus, in one aspect the present invention provides that the suncare formulation can be used in a method for protecting skin from UV damage comprising topically administering the suncare formulation to the skin (preferably, mammalian skin; more preferably, human skin).

Some embodiments of the present invention will now be described in detail in the following Examples.

### Synthesis S1: Silylated Maltodextrin

Ammonium chloride (412.4 mg, 0.05 eq) and Glucidex® 1 maltodextrin (DE 1 from Roquette) (25.0 g, 0.154 mol, 1.0 eq) were combined in a 2 CV Helicone mixer (CIT) with a stirring speed at 5 Hz. The resulting mixture was transferred to a reactor. Hexamethyldisilazane (80.87 g, 3.25 eq) was then added to the reactor contents dropwise. Dimethyl sulfoxide (10.8 g) was then added to the reactor contents. The reactor was then sealed and continuously flushed with nitrogen. The reactor contents were stirred at 20 Hz. Heat was applied to the reactor using a heating mantle set to a temperature of 40° C. and the stirring was increased to 50 Hz. After 30 min, the heating mantle was set to 50° C. The temperature setting for the heating mantle was then increased to 80° C. over the course of 1 h by 10° C. increments. The reactor contents were stirred for 1 hr after the temperature of the reactor contents reached 71° C. The heating mantle was then removed and the stirring was decreased to 25 Hz. When the reactor contents cooled to <50° C., stirring was stopped and 400 mL of ethyl acetate was added to the reactor contents. Stirring was then resumed at 25 Hz for 5 min. Stirring was then stopped and the organic layer was transferred to a collection jar. Ethyl acetate (100 mL) was then added to the reactor contents and stirring was resumed at 25 Hz for 5 min. Stirring was then stopped and the organic layer was transferred to the contents of the collection jar. The contents of the collection jar was transferred to a separatory funnel and washed with distilled water two times (2×250 mL). The organic layer was collected in an Erlenmeyer flask, and dried with sodium sulfate. The organic layer was then concentrated under vacuum to yield a fine white powder (˜46.3 g). The degree of substitution, DS, of the —Si(CH₃)₃ on the maltodextrin base polymer was determined by ¹H NMR to be 1.67.

### Synthesis S2: Silylated Maltodextrin

Ammonium chloride (454.4 mg, 0.05 eq) and Glucidex® 1 maltodextrin (DE 1 from Roquette) (27.0 g, 0.166 mol, 1.0 eq) were combined in a 2 CV Helicone mixer (CIT) with a stirring speed at 5 Hz. The resulting mixture was transferred to a reactor. Hexamethyldisilazane (87.34 g, 3.25 eq) was then added to the reactor contents dropwise. Dimethyl sulfoxide (11.66 g) was then added to the reactor contents. The reactor was then sealed and continuously flushed with nitrogen. The reactor contents were stirred at 20 Hz. Heat was applied to the reactor using a heating mantle set to a temperature of 92° C. and the stirring was increased to 50 Hz. The reactor contents were stirred for 1.5 hr after the temperature of the reactor contents reached 83° C. The heating mantle was then removed and the stirring was decreased to 25 Hz. When the reactor contents cooled to <50° C., stirring was stopped and 400 mL of ethyl acetate was added to the reactor contents. Stirring was then resumed at 25 Hz for 5 min. Stirring was then stopped and the organic layer was transferred to a collection jar. Ethyl acetate (100 mL) was then added to the reactor contents and stirring was resumed at 25 Hz for 5 min. Stirring was then stopped and the organic layer was transferred to the contents of the collection jar. The contents of the collection jar was transferred to a separatory funnel and washed with distilled water two times (2×250 mL). The organic layer was collected in an Erlenmeyer flask, and dried with sodium sulfate. The organic layer was then concentrated under vacuum to yield a fine white powder (˜56.6 g). The degree of substitution, DS, of the —Si(CH₃)₃ on the maltodextrin base polymer was determined by ¹H NMR to be 2.1.

### Synthesis S3: Silylated Maltodextrin

Ammonium chloride (445.4 mg, 0.05 eq) and Glucidex® 1 maltodextrin (DE 1 from Roquette) (27.0 g, 0.167 mol, 1.0 eq) were combined in a 2 CV Helicone mixer (CIT) with a stirring speed at 5 Hz. The resulting mixture was transferred to a reactor. Hexamethyldisilazane (60.47 g, 2.25 eq) was then added to the reactor contents dropwise. Dimethyl sulfoxide (11.7 g) was then added to the reactor contents. The reactor was then sealed and continuously flushed with nitrogen. The reactor contents were stirred at 20 Hz. Heat was applied to the reactor using a heating mantle set to a temperature of 55° C. and the stirring was increased to 50 Hz. After 5 min, the heating mantle was set to 102° C. The reactor contents were stirred for 2 hr. The heating mantle was then removed and the stirring was decreased to 25 Hz. When the reactor contents cooled to <50° C., stirring was stopped and 400 mL of ethyl acetate was added to the reactor contents. Stirring was then resumed at 25 Hz for 5 min. Stirring was then stopped and the organic layer was transferred to a collection jar. Ethyl acetate (100 mL) was then added to the reactor contents and stirring was resumed at 25 Hz for 5 min. Stirring was then stopped and the organic layer was transferred to the contents of the collection jar. The contents of the collection jar was transferred to a separatory funnel and washed with distilled water two times (2×250 mL). The organic layer was collected in an Erlenmeyer flask, and dried with sodium sulfate. The organic layer was then concentrated under vacuum to yield a fine white powder (˜407.3 g). The degree of substitution, DS, of the —Si(CH₃)₃ on the maltodextrin base polymer was determined by ¹H NMR to be 2.55.

### Synthesis S4: Silylated Maltodextrin

To a 25 mL scintillation vial was added ammonium chloride (33.0 mg, 0.05 eq) and Maltrin M250 maltodextrin (DE 23-27 from Grain Processing Corporation) (2.0 g, 12.3 mM, 1.0 eq). Hexamethyldisilazane (4.48 g, 2.25 eq) was then added dropwise to the vial contents. Dimethyl sulfoxide (1 g) was then added to the vial contents, and the vial was capped with a screw cap septum topped with two vent needles. The vial was placed on an aluminum heating block set at 85° C. for 1.5 hrs. The vial contents were then cooled down to <50° C. and diluted with ethyl acetate (150 mL). The organic layer was transferred to a separatory funnel and washed with distilled water three times (3×50 mL). The organic layer was collected in an Erlenmeyer flask, and dried with sodium sulfate. The organic layer was then concentrated under vacuum to yield a fine white powder (˜4.15 g). The degree of substitution, DS, of the —Si(CH₃)₃ on the maltodextrin base polymer was determined by ¹H NMR to be 2.2.

### Synthesis S5: Silylated Cellulose

Polysaccharide (BioFloc XV, 15.0 g, from Tartas) was weighed in a 2 L three-neck flask equipped with a nitrogen inlet and a temperature controller. Solvent (N,N dimethylacetamide, 331 g, from Sigma-Aldrich) was added and the reaction mixture was placed under an atmosphere of nitrogen with an outlet to avoid over-pressurization of the reactor. Silane (hexamethyldisilazane, 30 g, from The Dow Chemical Company) was added at once to the reaction mixture. The mixture was slowly heated to a set temperature of 130° C. and stirred for 7.5 h. The solution was cooled naturally, then xylenes (600 g, from Sigma-Aldrich) was added to the reaction mixture along with additional hexamethyldisilazane (20 g) and the mixture was stirred for 4 h with a set temperature of 125° C. The reactor contents were left to cool down to room temperature overnight. The reactor contents were then transferred to a separatory funnel and subjected to non-solvent precipitation by dropwise addition into 2 L of vigorously stirring methanol. The product was isolated by filtration, and dried in a vacuum oven at 50° C. overnight. The product was then suspended in 500 ml of methanol, then filtered and dried in a vacuum oven at 50° C. overnight. The product was analyzed by attenuated total reflection infrared to determine DS at 2.23.

### Synthesis S6: Silylated Cellulose

Polysaccharide (E-60, 15.2 g, from GP Cellulose) was weighed in a 2 L three-neck flask equipped with a nitrogen inlet and a temperature controller. Solvent (N,N dimethylacetamide, 324 g) was added and the reaction mixture was placed under an atmosphere of nitrogen with an outlet to avoid over-pressurization of the reactor. Silane (hexamethyldisilazane, 50.2 g, from The Dow Chemical Company) was added at once to the reaction mixture, along with the saccharin catalyst (850 mg, from Sigma-Aldrich). The mixture was slowly heated to a set temperature of 130° C. and stirred for 5 h. The solution was cooled naturally, then xylenes (400 g) were added to the reaction mixture and the mixture was stirred at 120° C. for 8 hours. The reactor contents were left to cool down to room temperature overnight. The cooled reactor contents were then transferred to a separatory funnel and subjected to non-solvent precipitation by dropwise addition into 2 L of vigorously stirring methanol. The product was isolated by filtration and dried in a vacuum oven at 50° C. overnight. The product was then suspended in 500 ml of methanol, then filtered again, and dried in a vacuum oven at 50° C. overnight. was analyzed by attenuated total reflection infrared to determine DS at 2.6.

### Synthesis S7: Silylated Maltodextrin

To a 25 mL scintillation vial was added ammonium chloride (33.0 mg, 0.05 eq) and Maltrin M200 maltodextrin (DE range 16.5-19.9 from Grain Processing Corporation)(2.0 g, 12.3 mM, 1.0 eq). Hexamethyldisilazane (3.58 g, 1.80 eq) was then added dropwise to the vial contents. Dimethyl sulfoxide (0.75 g) was then added to the vial contents and the vial was outfitted with a septum cap with two vent needles. The vial was placed on a heating block set at 80° C. for 1 hr. The vial contents were then cooled down to <50° C. and diluted with ethyl acetate (150 mL). The organic layer was transferred to a separatory funnel and washed with distilled water three times (3×50 mL). The organic layer was collected in an Erlenmeyer flask, and dried with sodium sulfate. The organic layer was then concentrated under vacuum to yield a fine white powder (˜3.6 g). The degree of substitution, DS, of the —Si(CH₃)₃ on the maltodextrin base polymer was determined by ¹H NMR to be 2.76.

### Synthesis S8: Silylated Maltodextrin

To a 25 mL scintillation vial was added ammonium chloride (33.0 mg, 0.05 eq) and Maltrin M040 (DE 4-7 from Grain Processing Corporation) (2.0 g, 12.3 mM, 1.0 eq). Hexamethyldisilazane (4.48 g, 2.25 eq) was then added dropwise to the vial contents. Dimethyl sulfoxide (1 g) was then added to the vial contents and the vial was outfitted with a septum cap with two vent needles. The vial was placed on a heating block set at 85° C. for 1.5 hrs. The vial contents were then cooled down to <50° C. and diluted with ethyl acetate (150 mL). The organic layer was transferred to a separatory funnel and washed with distilled water three times (3×50 mL). The organic layer was collected in an Erlenmeyer flask, and dried with sodium sulfate. The organic layer was then concentrated under vacuum to yield a fine white powder (˜4.15 g). The degree of substitution, DS, of the —Si(CH₃)₃ on the maltodextrin base polymer was determined by ¹H NMR to be 2.5.

### Synthesis S9: Silylated Maltodextrin

Ammonium chloride (445.4 mg, 0.05 eq) and Glucidex® 1 maltodextrin (DE 1 from Roquette) (27.0 g, 0.166 mol, 1.0 eq) were combined in a 2 CV Helicone mixer (CIT) with a stirring speed at 5 Hz. The resulting mixture was transferred to a reactor. Hexamethyldisilazane (87.34 g, 3.25 eq) was then added to the reactor contents dropwise. Dimethyl sulfoxide (11.66 g) was then added to the reactor contents. The reactor was then sealed and continuously flushed with nitrogen. The reactor contents were stirred at 20 Hz. Heat was applied to the reactor using a heating mantle set to a temperature of 50° C. and the stirring was increased to 50 Hz. After 20 min, the heating mantle was set to 100° C. The reactor contents were stirred for 2 hrs. The heating mantle was then removed and the stirring was decreased to 25 Hz. When the reactor contents cooled to <50° C., stirring was stopped and 400 mL of ethyl acetate was added to the reactor contents. Stirring was then resumed at 25 Hz for 5 min. Stirring was then stopped and the organic layer was transferred to a collection jar. Ethyl acetate (100 mL) was then added to the reactor contents and stirring was resumed at 25 Hz for 5 min. Stirring was then stopped and the organic layer was transferred to the contents of the collection jar. The contents of the collection jar was transferred to a separatory funnel and washed with distilled water two times (2×250 mL). The organic layer was collected in an Erlenmeyer flask, and dried with sodium sulfate. The organic layer was then concentrated under vacuum to yield a fine white powder (˜53 g). The degree of substitution, DS, of the —Si(CH₃)₃ on the maltodextrin base polymer was determined by ¹H NMR to be 2.23.

### Synthesis S10: Silylated Maltodextrin

To a 25 mL scintillation vial was added ammonium chloride (33.0 mg, 0.05 eq) and Glucidex® 1 maltodextrin (DE 1 from Roquette) (2.0 g, 12.3 mM, 1.0 eq). Hexamethyldisilazane (4.48 g, 2.25 eq) was then added dropwise to the vial contents. Dimethyl sulfoxide (1 g) was then added to the vial contents and the vial was outfitted with a septum cap with two vent needles. The vial was placed on a heating block set at 85° C. for 2 hrs. The vial contents were then cooled down to <50° C. and diluted with ethyl acetate (150 mL). The organic layer was transferred to a separatory funnel and washed with distilled water three times (3×50 mL). The organic layer was collected in an Erlenmeyer flask, and dried with sodium sulfate. The organic layer was then concentrated under vacuum to yield a fine white powder (˜3.96 g). The degree of substitution, DS, of the —Si(CH₃)₃ on the maltodextrin base polymer was determined by ¹H NMR to be 2.51.

### Synthesis S11: Silylated Maltodextrin

Ammonium chloride (445.4 mg, 0.05 eq) and Glucidex® 1 maltodextrin (DE 1 from Roquette) (27.0 g, 0.166 mol, 1.0 eq) were combined in a 2 CV Helicone mixer (CIT) with a stirring speed at 5 Hz. The resulting mixture was transferred to a reactor. Hexamethyldisilazane (87.34 g, 3.25 eq) was then added to the reactor contents dropwise. Dimethyl sulfoxide (11.66 g) was then added to the reactor contents. The reactor was then sealed and continuously flushed with nitrogen. The reactor contents were stirred at 20 Hz. Heat was applied to the reactor using a heating mantle set to a temperature of 50° C. and the stirring was increased to 50 Hz. After 20 min, the heating mantle was set to 100° C. The reactor contents were stirred for 2 hrs. The heating mantle was then removed and the stirring was decreased to 25 Hz. When the reactor contents cooled to <50° C., stirring was stopped and 400 mL of ethyl acetate was added to the reactor contents. Stirring was then resumed at 25 Hz for 5 min. Stirring was then stopped and the organic layer was transferred to a collection jar. Ethyl acetate (100 mL) was then added to the reactor contents and stirring was resumed at 25 Hz for 5 min. Stirring was then stopped and the organic layer was transferred to the contents of the collection jar. The contents of the collection jar was transferred to a separatory funnel and washed with distilled water two times (2×250 mL). The organic layer was collected in an Erlenmeyer flask, and dried with sodium sulfate. The organic layer was then concentrated under vacuum to yield a fine white powder (˜56.3 g). The degree of substitution, DS, of the —Si(CH₃)₃ on the maltodextrin base polymer was determined by ¹H NMR to be 2.42.

### Synthesis 512: Silylated Maltodextrin

To a 25 mL scintillation vial was added ammonium chloride (24.7 mg, 0.05 eq) and dried Glucidex® 1 maltodextrin (DE 1 from Roquette) (1.5 g, 3.25 eq). Hexamethyldisilazane (4.85 g, 3.25 eq) was then added dropwise to the vial contents. Dimethyl sulfoxide (0.8 g) was then added to the vial contents and the vial was outfitted with a septum cap with two vent needles. The vial was placed on a heating block set at 90° C. for 4 hrs. The vial contents were then cooled down to <50° C. and diluted with ethyl acetate (200 mL). The organic layer was transferred to a separatory funnel and washed with a 50/50 vol/vol mixture of brine and distilled water three times (3×60 mL). The organic layer was collected in an Erlenmeyer flask, and dried with sodium sulfate. The organic layer was then concentrated under vacuum to yield an off white crystalline solid, that was easily reduced to a fine powder with a spatula. The product powder was dried under vacuum in an oven at 50° C. for 5 hrs. The degree of substitution, DS, of the —Si(CH₃)₃ on the maltodextrin base polymer was determined by ¹H NMR to be 2.42.

### Synthesis S13: Silylated Maltodextrin

To a 25 mL scintillation vial was added ammonium chloride (24.7 mg, 0.05 eq) and dried Maltrin M150 maltodextrin (DE 13-17 from Grain Processing Corporation)(1.5 g, 1.0 eq). Hexamethyldisilazane (4.85 g, 3.25 eq) was then added dropwise to the vial contents. Dimethyl sulfoxide (0.8 g) was then added to the vial contents and the vial was outfitted with a septum cap with two vent needles. The vial was placed on a heating block set at 90° C. for 2.5 hrs. The vial contents were then cooled down to <50° C. and diluted with ethyl acetate (200 mL). The organic layer was transferred to a separatory funnel and washed with a 50/50 vol/vol mixture of brine and distilled water three times (3×60 mL). The organic layer was collected in an Erlenmeyer flask, and dried with sodium sulfate. The organic layer was then concentrated under vacuum to yield an off white crystalline solid, that was easily reduced to a fine powder with a spatula. The product powder was dried under vacuum in an oven at 50° C. for 5 hrs. The degree of substitution, DS, of the —Si(CH₃)₃ on the maltodextrin base polymer was determined by ¹H NMR to be 2.64.

### Synthesis S14: Silylated Maltodextrin

To a 25 mL scintillation vial was added ammonium chloride (33.0 mg, 0.05 eq) and Maltrin M200 maltodextrin (DE 20-23 from Grain Processing Corporation)(2.0 g, 1.0 eq). Hexamethyldisilazane (3.58 g, 1.80 eq) was then added dropwise to the vial contents. Dimethyl sulfoxide (0.75 g) was then added to the vial contents and the vial was outfitted with a septum cap with two vent needles. The vial was placed on a heating block set at 80° C. for 1 hrs. The vial contents were then cooled down to <50° C. and diluted with ethyl acetate (150 mL). The organic layer was transferred to a separatory funnel and washed with a 50/50 vol/vol mixture of brine and distilled water three times (3×50 mL). The organic layer was collected in an Erlenmeyer flask, and dried with sodium sulfate. The organic layer was then concentrated under vacuum to yield a fine white powder. The product powder was dried under vacuum in an oven at 50° C. for 5 hrs. The degree of substitution, DS, of the —Si(CH₃)₃ on the maltodextrin base polymer was determined by ¹H NMR to be 2.45.

### Solubility Screening (2 wt %)

The solubility of the products of Syntheses S1-S7 were assessed in different carriers by combining individually in separate vials the products of Syntheses S1-S7 (0.1 g) with various solvents (4.9 g) as noted in TABLE 1. The resulting 2 wt % solutions were stirred with a magnetic stir bar for 1 hour at ˜22° C. The carriers and solubility observations are provided in TABLE 1.

TABLE 1

| | Observation | | | | | | |
|---|---|---|---|---|---|---|---|
| | Solute (2 wt %) in carrier | | | | | | |
| Carrier | S1 | S2 | S3 | S4 | S5 | S6 | Malt⁹ |
| Water | I | I | I | I | I | I | S |
| Ethanol | I | S | S | S | I | I | I |
| Dimethicone, 2 cSt¹ | — | S | S | S | I | S* | I |
| Isododecane | I | S | S | S | I | I | I |
| Isohexadecane | — | S | S | — | — | — | — |
| Methyl undecanoate | — | S | S | S | — | — | — |
| Triglycerides² | — | S | S | S | — | — | — |
| Dicaprylyl carbonate³ | — | S | S | S | — | — | — |
| C₁₂₋₁₅ alkyl benzoate⁴ | — | S | S | S | I | I | I |
| C₁₃₋₁₅ Alkane⁵ | — | S | S | S | — | — | — |
| Dioctylether | — | S | S | S | — | — | — |
| Ethyl PG-Acetal | — | S | S | — | — | — | — |
| Levulinate⁶ | | | | | | | |
| Homosalates⁷ | — | S | S | S | — | — | — |
| Sunflower oil⁸ | — | I | I | S* | I | I | I |

¹DOWSIL ™ 200 fluid (2 cSt) from The Dow Chemical Company
²CRODAMOL ™ GTCC from Croda
³CETIOL ® CC from BASF
⁴CRODAMOL ™ ABLQ from Croda
⁵NEOSSANCE ® hemisqualane from Centerchem
⁶ECOSMOOTH ™ universal fluid 1100 from The Dow Chemical Company
⁷PARSOL ® HMS from DSM
⁸Sunflower oil, USDA Certified Organic from MakingCosmetics
⁹Glucidex ® 1 maltodextran with DE 1 from Roquette.
S Soluble and clear
S* Soluble and slightly hazy
I Insoluble

### Solubility Screening (50 wt %)

The solubility of the products of Syntheses S2-S3 and S7 (2 g) were assessed in isododecane (2 g) as noted in TABLE 2. The resulting 50 wt % solutions were stirred with a magnetic stir bar for 1 hour at ˜22° C. The carriers and solubility observations are provided in TABLE 2.

TABLE 2

Observation
Solute (50 wt %) in carrier

| Carrier | S2 | S3 | S7 |
|---|---|---|---|
| Isododecane | S | S | S |
| Ethanol | not tested | S | S |

S Soluble and clear

### Viscosity in Isododecane

The product of Synthesis S5-S6 and S9 was dissolved in isododecane at different concentrations in as noted in TABLE 3. The viscosity of the resulting solutions were then determined using a Brookfield DV-111-ultra viscometer, equipped with a SC4-28 spindle at ˜22° C. and 100 rpm. The results are provided in TABLE 3.

TABLE 3

| Solute | Concentration (wt %) | Viscosity (cP) |
|---|---|---|
| Synthesis S5 | 2 | 3,500 |
| Synthesis S6 | 2 | 3,200 |
| Synthesis S6 | 5 | gel |
| Synthesis S9 | 2 | 0 |
| Synthesis S9 | 5 | 2 |
| Synthesis S9 | 20 | 5 |
| Synthesis S9 | 40 | 305 |
| Synthesis S9 | 50 | 1,260 |

### Comparative Examples CF1-CF6 and Examples F1-F4: Suncare Formulations

Suncare formulations were prepared having compositions as noted in TABLE 4. The avobenzone, caprylic/capric triglyceride and ethylhexyl salicylate were mixed in a flask and heated to 60° C. until all the avobenzone was melted. The heat source was removed and the rest of the ingredients other than the ethanol were added to the contents of the flask. When the flask contents cooled to <30° C., the ethanol was added with stirring for 30 minutes until the flask contents were homogeneous.

TABLE 4

| | CF1 | CF2 | CF3 | CF4 | CF5 | CF6 | F1 | F2 | F3 | F4 |
|---|---|---|---|---|---|---|---|---|---|---|
| Ingredient | wt % | | | | | | | | | |
| Ethanol | q.s. 100 | | | | | | | | | |
| Caprylic/Capric | 5 | 5 | 5 | 5 | 5 | 5 | 5 | 5 | 5 | 5 |
| Triglyceride¹ | | | | | | | | | | |
| Homosalate² | 5 | 5 | 5 | 5 | 5 | 5 | 5 | 5 | 5 | 5 |
| Ethylhexyl salicylate³ | 5 | 5 | 5 | 5 | 5 | 5 | 5 | 5 | 5 | 5 |
| Avobenzone⁴ | 3 | 3 | 3 | 3 | 3 | 3 | 3 | 3 | 3 | 3 |
| Octocrylene⁵ | 4 | 4 | 4 | 4 | 4 | 4 | 4 | 4 | 4 | 4 |
| Ethyl cellulose⁶ | — | 4 | — | — | — | — | — | — | — | — |
| Product Synthesis S1 | — | — | — | 4 | — | — | — | — | — | — |
| Product Synthesis S3 | — | — | — | — | — | — | — | 4 | — | — |
| Product Synthesis S6 | — | — | 4 | — | — | — | — | — | — | — |
| Product Synthesis S9 | — | — | — | — | — | — | 4 | — | — | — |
| Product Synthesis S13 | — | — | — | — | — | — | — | — | — | 4 |
| Product Synthesis S8 | — | — | — | — | — | — | — | — | 4 | — |
| Product Synthesis S7 | — | — | — | — | 4 | — | — | — | — | — |
| Product Synthesis S14 | — | — | — | — | — | 4 | — | — | — | — |

¹From Rita under tradename Ritamollient CCT
²From DSM under tradename PARSOL ® HMS
³From DSM under tradename PARSOL ® EHS
⁴From DSM under tradename PARSOL ® 1789
⁵From DSM under tradename PARSOL ® 340
⁶From DuPont under tradename ETHOCEL ™ Standard 7 Premium

### Suncare Formulation Clarity

Suncare formulations prepared according to Comparative Examples CF1-CF3 and Example F3 were observed for clarity. The observations are provided in TABLE 5.

### Viscosity

The viscosity of the suncare formulations prepared according to Comparative Examples CF1-CF3 and Example F1 were determined using a TA Instruments DHR-3 rheometer at ˜22° C., equipped with spindle 27 rotated at 250 rpm. The measured viscosities are reported in TABLE 5.

TABLE 5

| Example | Observation | Viscosity (cP) |
|---|---|---|
| Compative Example CF1 | A | <5 |
| Compative Example CF2 | A | <5 |
| Compative Example CF3 | B | <5 |
| Example F1 | A | <5 |

A Clear, homogeneous
B Clear with sedimentation, not fully soluble

### In-Vitro SPF Performance

The suncare formulations prepared according to Comparative Examples CF1-CF2, CF4-CF6 and Examples F1-F4 were evaluated for SPF performance. The SPF performance of each of the suncare formulations was then tested in triplicate using an in-vitro technique according to the following protocol.

The substrate used for the in-vitro SPF measurements was a rough PMMA substrate (6 μm—HD6 available from Schonberg GMBH & Co. KG). The suncare formulations to be tested were each applied to three separate rough 5 cm×5 cm PMMA substrates using an RDS #7 wire draw down bar to provide a uniform layer of the suncare formulation over the surface of the PMMA substrate at a rate of 1.3 mg/cm². Each deposited layer of suncare formulation was allowed to dry for (60) minutes under ambient conditions in the laboratory. The UV absorption of each dried layer of suncare formulation between 290 nm and 400 nm was then measured at nine (9) separate points using a Labsphere UV-2000S Spectrometer. The in-vitro SPF value for each suncare formulation prepared according to Comparative Examples CF1-CF2, CF4-CF6 and Examples F1-F4 was then calculated based on the results of the UV absorption measurements. The average from the triplicate samples of each suncare formulation prepared according to Comparative Examples CF1-CF2, CF4-CF6 and Examples F1-F4 is reported in in TABLE 6.

TABLE 6

| | | | | % SPF |
|---|---|---|---|---|
| Suncare Formulation | DE | DS | in-vitro SPF | Deviation |
| Comp. Ex. CF1 | — | — | 18.8 | 0 |
| Comp. Ex. CF2 | — | — | 25.6 | 35 |
| Comp. Ex. CF4^{a} | 1 | 1.68 | 21.8 | 16 |
| Comp. Ex. CF5 | 16.9-19.9 | 2.76 | 15.2 | −19 |
| Comp. Ex. CF6 | 20-23 | 2.86 | 14.5 | 14 |
| Example F1 | 1 | 2.23 | 36.6 | 95 |
| Example F2 | 1 | 2.55 | 25.4 | 35 |
| Example F3 | 4-7 | 2.48 | 28.8 | 53 |
| Example F4 | 13-17 | 2.69 | 23.9 | 27 |

^{a}Formulation hazy with visible sediments

We claim:

1. An SPF booster, comprising:
a functionalized maltodextrin comprising a maltodextrin base polymer functionalized with —Si(R¹)₃ groups; wherein each R¹ is independently a C₁₋₁₀ linear or branched, saturated alkyl group; wherein the maltodextrin base polymer has a dextrose equivalent, DE, of 1 to 15; wherein the functionalized maltodextrin has a degree of substitution of —Si(R¹)₃ groups, DS, of 1.7 to 3; and wherein the functionalized maltodextrin is free of vinylic carbon.

2. A suncare formulation, comprising:
a dermatologically acceptable organic carrier;
a UV radiation absorbing agent; and
an SPF booster according to claim 1.

3. The suncare formulation according to claim 2, wherein the dermatologically acceptable organic carrier is selected from the group consisting of a glycol (e.g., ethylene glycol, propylene glycol, butylene glycol, pentylene glycol, hexylene glycol, dipropylene glycol, ethoxydiglycol), a C₁₋₄ straight or branched chain alcohol (e.g., methyl alcohol, ethyl alcohol, propyl alcohol, isopropyl alcohol, butyl alcohol), acetone, methyl acetate, butyl cellusolve, dimethicone, isododecane, isohexadecane, methyl undecanoate, dermatologically acceptable hydrophobic ester oil (e.g., caprylic capric triglycerides), dicaprylyl carbonate, C₁₂₋₁₅ alkyl benzoate, hemisqualane, dioctylether, levulinic acid and mixtures thereof.

4. The suncare formulation according to claim 3, wherein the UV radiation absorbing agent is selected from the group consisting of physical blockers, chemical absorbers and mixtures thereof.

5. The suncare formulation according to claim 3, wherein the UV radiation absorbing agent is selected from the group consisting of red petrolatum; titanium dioxide; zinc oxide; 1-(4-methoxyphenol)-3-(4-tert-butylphenyl)propane-1,3-dione; 2-hydroxy-4-methoxybenzophenone; dioxybenzone; sulisobenzone; menthyl anthranilate; para-aminobenzoic acid; amyl paradimethylaminobenzoic acid; octyl para-dimethylaminobenzoate; ethyl 4-bis(hydroxypropyl) para-aminobenzoate; polyethylene glycol (PEG-25) para-aminobenzoate; ethyl 4-bis(hydroxypropyl) aminobenzoate; diethanolamine para-methyoxycinnamate; 2-ethoxyethyl para-methoxycinnamate; ethylhexyl para-methoxycinnamate; octyl para-methoxycinnamate; isoamyl para-methoxycinnamate; 2-ethylhexyl-2-cyano-3,3-diphenyl-acrylate; 2-ethylhexyl-2-cyano-3,3-diphenyl-2-propenoate; 2-ethylhexyl-2-hydroxybenzoate; homomenthyl salicylate; glyceryl aminobenzoate; triethanolamine salicylate; digalloyl trioleate; lawsone with dihydroxyacetone; 2-phenylbenzimidazole-5-sulfonic acid; 4-methylbenzylidine camphor; avobenzone; triazines; benzotriazoles; vinyl group-containing amides; cinnamic acid amides; sulfonated benzimidazoles); 3,3,5-trimethylcyclohexyl 2-hydroxybenzoate and mixtures thereof.

6. The suncare formulation according to claim 3, wherein the UV radiation absorbing agent is a mixture of UV absorbing agents including 1-(4-methoxyphenol)-3-(4-tert-butylphenyl)propane-1,3-dione; 2-ethylhexyl 2-hydroxybenzoate; 2-ethyhexyl-2-cyano-3,3-diphenyl-2-propenoate and 3,3,5-trimethylcyclohexyl 2-hydroxybenzoate.

7. The suncare formulation according to claim 6, wherein the dermatologically acceptable organic carrier includes a C₁₋₄ straight or branched chain alcohol.

8. The suncare formulation according to claim 7, wherein the dermatologically acceptable organic carrier includes ethanol.

9. The suncare formulation of claim 8, further comprising a dermatologically acceptable hydrophobic ester oil.

10. A method of protecting skin from exposure to the sun, comprising:
providing a suncare formulation according to claim 2, and
applying the suncare formulation to skin.
